(19) United States
(12) Patent Application Publication
(10) Pub. No.: US 2006/0049270 A1
(43) Pub. Date: Mar. 9, 2006

(54) Tower device for supporting and displaying aromatic tins

(76) Inventor: Peter Wayne, Northville, MI (US)

Correspondence Address: GIFFORD, KRASS, GROH, SPRINKLE & CITKOWSKI, P.C, PO BOX 7021, TROY, MI, 48007-7021, US

(21) Appl. No.: 11/214,187

(22) Filed: Aug. 29, 2005

Related U.S. Application Data
(60) Provisional application No. 60/605,448, filed on Aug. 30, 2004.

(51) Int. Cl. A61M 11/04; B01F 3/04

(52) U.S. Cl. 239/57; 261/DIG. 88

(57) ABSTRACT

An elevated structure for supporting a plurality of scenting compositions. The tower is secured upon a rotatable base and includes a plurality of individual levels. A plurality of scented tins are each releasably secured to a given level associated with the tower. The tower further includes a metallic composition, such that each of the tins further includes a magnet for securing at a location associated with the tower. A frictional (e.g. rubberized) bottom is associated with the base and supports the tower upon a level surface, such as to further permit the tower to be rotated.

### CROSS REFERENCE TO RELATED APPLICATIONS

The present application claims the priority of U.S. Provisional Patent Application Ser. No. 60/605,448, filed Aug. 30, 2004, and entitled “Tower Device for Supporting and Displaying Aromatic Tins.”

### BACKGROUND OF THE INVENTION

1. Field of the Invention

The present invention is directed to a tower device for supporting any plurality of fragrancing/aroma tins, and such as in particular having a rotatable base and which may be associated with a plurality of individually magnetically attachable and seasonal-theme fragrances.

2. Description of the Prior Art

The prior art is well documented with various types of fragrancing or scenting devices. The objective in each instance is to provide a mechanism for issuing a selected scent, such as for purposes of aromatherapy and the like.

A first example drawn from the prior art is set forth in U.S. Pat. No. 6,581,915, issued to Bartsch, and which teaches a dispensing device for dispensing scents into the environment. The dispensing device is used in conjunction with an article of manufacture which contains one or more scents or aromatic materials. In such an embodiment, the dispensing device can include a housing and a removable, reusable/replayable, closed and multiple scent-containing article of manufacture which is removably inserted into or onto the housing.

Schwartz, U.S. Pat. No. 5,805,768, teaches an aromatherapy diffuser for use with aromatherapy oils, and which allows the user to preselect a variety of aromas to be introduced at predetermined time intervals so that different moods or state of minds may be created. The apparatus includes a tray having a plurality of receptacles for various aromatic materials, and a heating means for heating a preselected receptacle filled with an aromatic material. The apparatus also includes a motor driven timer which rotates the tray so that the plurality of receptacles containing different aromatic materials are exposed to the heating means for predetermined time periods. The apparatus further includes a lid with a hole that exposes the preselected receptacle and aromatic material when the receptacle and aromatic material are exposed to the heating means, and further such that the aroma released from the heated aromatic material emanates into the environment through the associated hole.

Yip, U.S. Pat. No. 6,859,615, teaches a multi-fragrance scent dispenser including a housing having an outlet opening; first and second holders in the housing for holding first and second liquid fragrance containers having first and second wicks, respectively, extending out of the first and second liquid fragrance containers; a first heater positioned in the housing to be in surrounding relation to the first wick when the first liquid fragrance container is held by the first holder; a second heater positioned in the housing to be in surrounding relation to the second wick when the first liquid fragrance container is held by the first holder; a control circuit which selectively controls activation of the first and second heaters; and a blocking plate movable relative to the heaters for at least partially blocking escape of evaporated fragrance through the outlet opening from at least one of the first and second wicks.

Spector, U.S. Pat. No. 4,629,604, teaches a multi-aroma cartridge player exhibiting a planar like array of frame assemblies held within a multi-section framework. Each assembly is formed by a pad of absorbent material sandwiched between a pair of frames whose margins are joined together to define a central zone exposing the pad. Each pad is impregnated with a different liquid fragrance. Upon the cartridge being inserted in a slot defined in the player case, it overlies a complementary honeycomb, each of whose cells is then positioned in registration with a respective assembly. Each of the cells are provided with individual heaters such that when a selected cell heater is energized, it heats the air in the cell to produce a positive pressure therein that acts to force the heated air through the zone to volatize the liquid fragrance, the resulting aromatic vapor being discharged into the atmosphere through vents in the case. The selection of aromas to be played may be effected manually or may be synchronized to follow the scenes of a video tape or movie film presentation.

U.S. Pat. No. 5,023,020, issued to Machida et al., teaches a method for supplying aromas to a space which includes the steps of retaining a plurality of aromatic materials in respective reservoirs, selecting at least one of the aromatic materials according to a predetermined timetable determined on the basis of activities of people in the space at each time, diffusing a predetermined amount of the selected aromatic material in air to obtain aromatized air, and sending the aromatized air into the surrounding space. The apparatus includes a receptor having plural aromas, a controller for controlling the receptor to select one stated aroma from the plural aromas in the receptor.

Finally, Japanese Patent Abstract 10057464A2 teaches an electric aromatization device including a cup-like metallic container to which a columnar metallic magnet 15 is fixed through a planar heat panel mounted to the upper part of an electric heating body. The dome includes several ventilation holes formed at intervals on the upper part of the cup-like metallic container. A motor-driven fan is connected to a lower end of an interiorly suspended column and for assisting in the dispersal of the desired scenting medium.

### SUMMARY OF THE PRESENT INVENTION

The present invention discloses an elevated structure for supporting a plurality of scenting compositions. The tower is secured upon a rotatable base and includes a plurality of individual levels. A plurality of scented tins are each releasably secured to a given level associated with the tower.

The tower further includes a metallic composition, such that each of the tins further includes a magnet for securing at a location associated with the tower. A frictional (e.g. rubberized) bottom is associated with the base and supports the tower upon a level surface, such as to further permit the tower to be rotated. Alternatively, rubberized pad supports may provide for supporting the tower in secure and freely rotative fashion.

In one variant, the tower includes a plurality of planar supports (levels), arranged in spatially arrayed and separated fashion by a plurality of generally vertically extending columns. In this variant, magnets are associated with upper edge locations of each of the tins are releasably secured in abutting fashion underneath a selected support level and in order to array the tins upon a given level and angular orientation.

In a further desired variant, the tower exhibits a substantially unitary structure and which includes a multiple (three) sided configuration. A plurality of scenting tins are each secured against a given face of the structure, typically again by a magnet secured to a rear side of the associated tin. The shape and configuration of the unitary construction, such as which may include a triangular or square shaped cross section, is typically hollowed on the inside and may include a removable top to permit storing a plurality of the individual scenting tins. In either variant, a figurine may be magnetically secured to a level associated with the tower.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a perspective view of the fragrancing tower design according to a preferred embodiment of the present invention;

FIG. 2 is a sectional view of a tin associated with the tower design of the present invention;

FIG. 3 is an exploded view of a scenting/fragrancing tower according to a further preferred embodiment of the present invention;

FIG. 4 illustrates an underside of a Lazy Susan (rotatable) base associated with the present invention; and

FIG. 5 is an illustration in perspective of a fragrancing tower design according to a further preferred embodiment of the present invention.

### DETAILED DESCRIPTION OF THE PREFERRED EMBODIMENTS

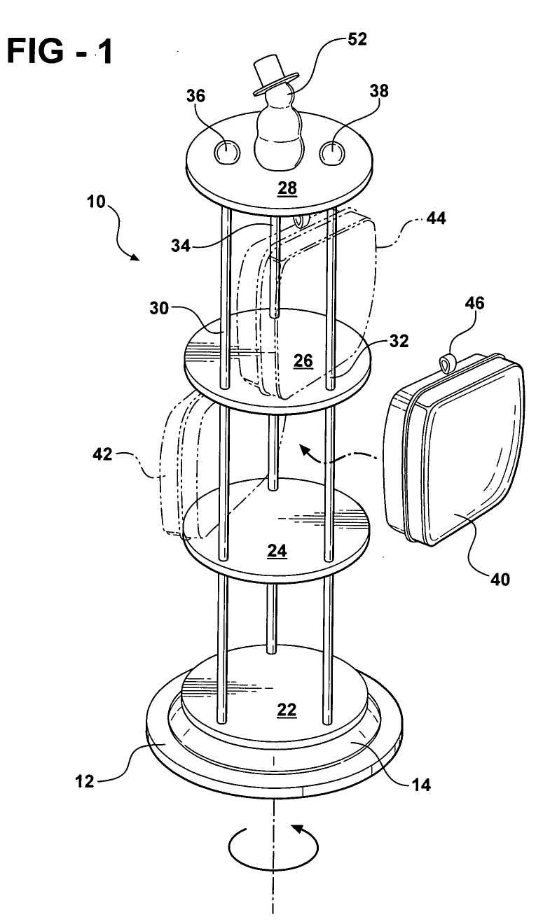

Referring now to FIG. 1, a fragrancing/aroma tower is illustrated at 10 according to a first preferred embodiment of the present invention. The tower includes a rotatable base 12, in one instance constructed of a durable (metallic) material having a circular cross section with a smooth faced top 14. As further illustrated in the exploded view of FIG. 3, a magnet 16 can be attached to the top 14 of the base and, referring further to the rotated underside view of FIG. 4, a bearing supported and rotatable plate 18 is frictionally secured upon a level surface by such as a non-slip material 20. Alternatively, rubberized pad supports 21 may support the tower upon the level surface in secure and rotatable fashion.

The use of metal is desired in the construction of the base, owing to its magnetically attractive properties, however it is also understood that other materials, such as a durable plastic, can be substituted. In the instance of a non-metal base, a magnet can be glued or otherwise mechanically securing to the top face of the base 12. It is also envisioned that, in addition to a circular shape, other cross-sectional shapes, include triangular, square, hexagonal, and so on, can be employed within the scope of the invention.

An upwardly extending structure associated with the tower 10 sets upon the base 12, such as by attaching to the magnet 16, and includes a plurality of individual levels. In particular, and referencing again FIG. 1, planar (disk shaped) supports are illustrated at 22, 24, 26 and 28, and which are supported in spatially arrayed fashion by vertical columns 30, 32 and 34. The columns 30, 32 and 34 can also be screwed together at individual levels (such as through the use of interengageable post extensions) and may further include tapped ball knobs (such as evident at 36 and 38 in FIG. 1) secured to a top face of the upper most support 28.

In a preferred embodiment, the lowermost support 22 is magnetically attached atop the rotatable base 12 (such as upon magnet 16). It is also envisioned that the tower can be secured in other fashion, and such as mechanically (glue, welding, adhesives).

A plurality of fragrant/aromatic tins are provided, and such as which are illustrated at 40, 42, 44, et seq. in FIG. 1. The tins each further include a magnet, see at 46 for magnet 40 in FIG. 1, such as which is secured to a top edge of the tin, and which releasably secures the tin to a selected location associated with a given tier of the structure.

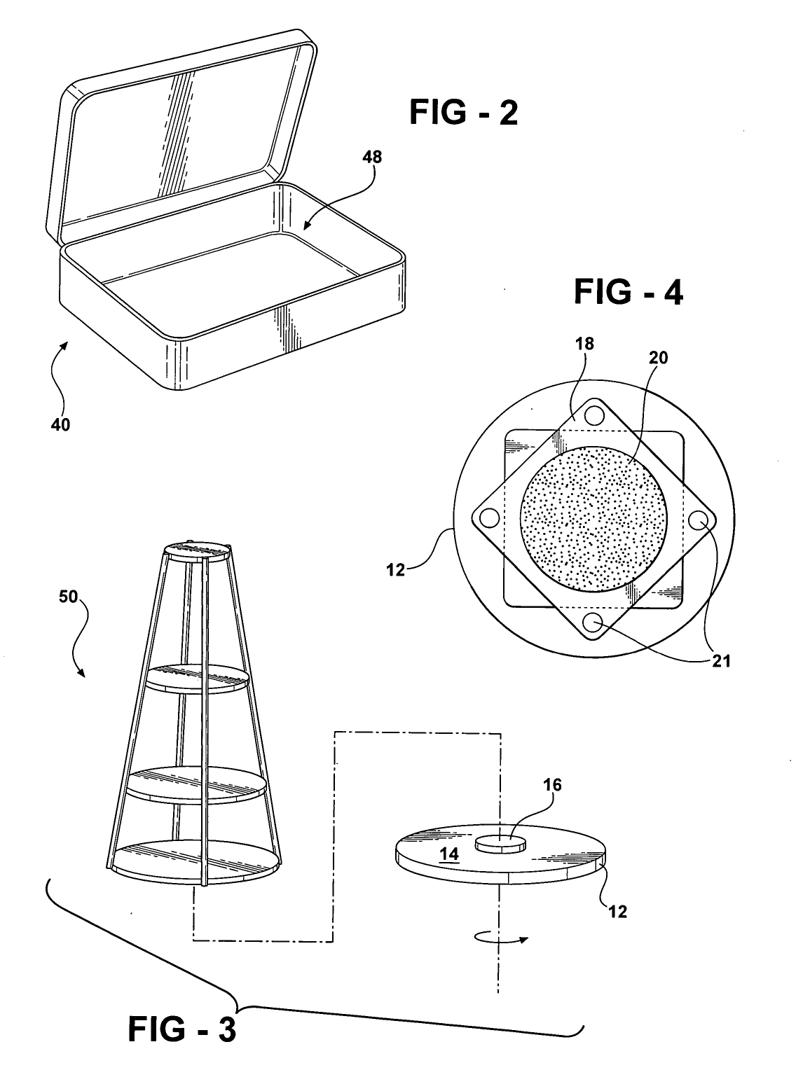

As best shown in FIG. 2, an inside of a selected tin 40 may include a foam composition 48, and such as which may be soaked or otherwise impregnated with a suitable scenting composition. The present invention contemplates the application of a number of different scenting/fragrancing compositions, and such as which may further be directed to different seasonal applications. It is also envisioned that different shaped fragrant holding containers, including other than scenting impregnated tins, can be utilized within the scope of the present invention.

Referring also to FIG. 3, an alternate configuration of an attachable scenting tower is illustrated at 50 and includes an inwardly sloped configuration, and as opposed to the generally planar configuration of the tower in the illustration 10 of FIG. 1. As also shown in FIG. 1, a decorative figurine 52 can be releasably and magnetically attached to any level associated with the elevated tower and it is further envisioned that differently configured figurines can be employed and such as for different holiday themes.

In each instance, the present invention provides the ability to conveniently collect, arrange, display, showcase and/or index a variety of different fragrance/aroma tins. The primary embodiments illustrated allow for up to three tins to be magnetically secured at each level and the base is further rotatable about the Lazy Susan arrangement (see again FIG. 4).

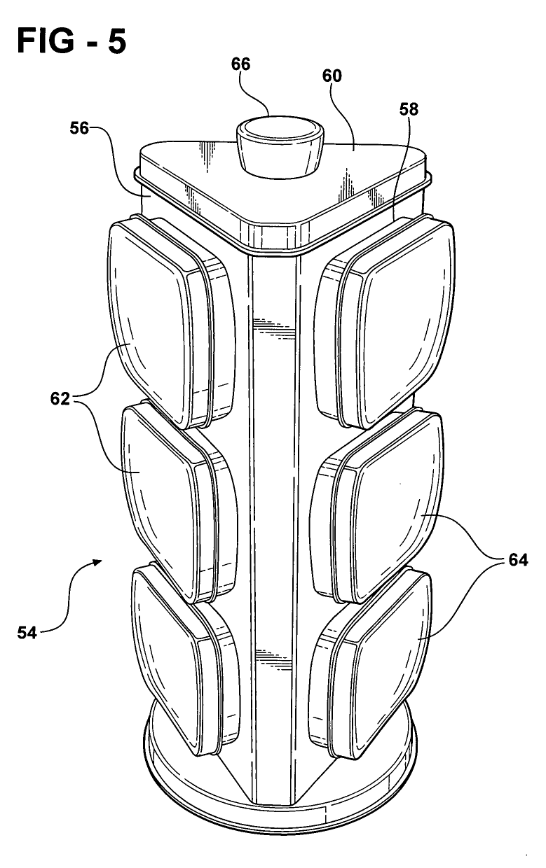

In a further desired variant, referring finally to FIG. 5, a tower 54 exhibits a substantially unitary structure and which includes a multiple (three) sided configuration, see sides 56, 58 and 60. It is further envisioned that the tower 54 can further exhibit such as a square shape, pentagon shape, and the like.

As with the previously described embodiment, pluralities of scenting tins, see at 62 and 64, are each secured against a selected one of the three faces 56, 58 and 60 of the tower structure 54, typically again by a magnet secured to a rear side of the associated tin. In a preferred variant, pluralities of three scenting tins are provided upon each of the three angularly offset faces. The shape and configuration of the unitary construction, and given its number of sides, is typically hollowed on the inside and may include a knob 66 configured with a removable top, permitting storage of a plurality of the individual scenting tins in any desired fashion.

It is therefore evident that the present invention discloses a novel and improved structure for supporting a plurality of individualized scenting compositions. Having described my invention, other and additional preferred embodiments will become apparent to those skilled in the art to which it pertains and without deviating from the scope of the appended claims.

I claim:

1. An elevated structure for supporting a plurality of scenting compositions, comprising:
a rotatable base;
a tower secured to said base and including a plurality of individual levels; and
a plurality of scented tins, each of said tins being releasably secured to a given level associated with said tower.

2. The structure as described in claim 1, said tower further including a metallic composition, each of said tins further comprising a magnet for securing at a location associated with said tower.

3. The structure as described in claim 1, further comprising a frictional bottom surface associated with said base.

4. The structure as described in claim 1, said tower further comprising a plurality of planar supports supported in spatially arrayed fashion by a plurality of generally vertically extending columns.

5. The structure as described in claim 1, said tower having a specified shape and size and being magnetically secured atop said base.

6. The structure as described in claim 1, further comprising a figurine magnetically secured to a level associated with said tower.

7. The structure as described in claim 1, said tower further comprising a substantially unitary structure exhibiting a plurality of interconnecting and angularly offset faces.

8. The structure as described in claim 7, said tower further comprising a three-sided configuration.

9. The structure as described in claim 7, said unitary structure further defining an interiorly hollowed cavity accessible by a removable top.

10. An elevated structure for supporting a plurality of scenting compositions, comprising;
a rotatable base exhibiting a frictional bottom surface;
a unitary structure exhibiting at least first, second and third interconnecting and angularly offset faces, said structure being secured in upwardly extending fashion upon said base; and
a plurality of scented tins, each of which are releasably secured to a given face of said structure.

11. The structure as described in claim 10, said tower further including a metallic composition, each of said tins further comprising a magnet for securing at a location associated with said tower.

12. The structure as described in claim 10, further comprising a frictional bottom surface associated with said base.

13. The structure as described in claim 12, said tower further comprising a three-sided configuration.

14. The structure as described in claim 13, said unitary structure further defining an interiorly hollowed cavity accessible by a removable top.
